(19) United States
(12) Patent Application Publication
(10) Pub. No.: US 2024/0099998 A1
(43) Pub. Date: Mar. 28, 2024

(54) COMPOSITIONS AND METHODS FOR INDUCING DEFECATION

(71) Applicant: Dignify Therapeutics, LLC, Research Triangle Park, NC (US)

(72) Inventors: Karl Bruce THOR, Cary, NC (US); Lesley MARSON, Chapel Hill, NC (US)

(21) Appl. No.: 18/269,973

(22) PCT Filed: Jan. 13, 2022
(86) PCT No.: PCT/US2022/012220

§ 371 Date: Jun. 28, 2023

Related U.S. Application Data
(60) Provisional application No. 63/137,778, filed on Jan. 15, 2021.

(51) Int. Cl. A61K 31/165; A61K 9/00; A61K 31/164; A61P 1/10

(52) U.S. Cl. CPC A61K 31/165; A61K 9/0031; A61K 31/164; A61P 1/10

(57) ABSTRACT

Methods, pharmaceutical formulations, and kits are provided for using Transient Receptor Potential Vanilloid 1 (TRPV1) agonists to increase rectal pressure and induce defecation in mammals.

### CROSS-REFERENCE TO RELATED APPLICATION

This application claims the benefit of priority of U.S. provisional patent application No. 63/137,778, filed on Jan. 15, 2021, the disclosure of which is incorporated herein by this reference in its entirety.

### TECHNICAL FIELD

The presently disclosed subject matter relates to methods of using transient receptor potential vanilloid 1 (TRPV1) receptor agonists for inducing defecation.

### BACKGROUND

Feces are formed in the large intestine and temporarily stored in the rectum. In healthy individuals, the timing of defecation is under voluntary control, mediated by the brain, spinal cord and peripheral nerves supplying the rectum. As feces fill and expand the rectum, stretch receptors in the rectal wall initiate a reflex contraction of rectal smooth muscle and relaxation of the internal anal sphincter (i.e. the rectoanal inhibitory reflex). During defecation, an increase in intrarectal pressure expands the anal canal, allowing the feces to pass through the anus (see FIG. 1). As shown in FIG. 1, defecation is initiated by stimulation of stretch receptors and afferent terminals in the rectal wall that produces simultaneous rectal contraction rostral to the stimulus and inhibition/relaxation of the anal sphincter.

The inability to voluntarily eliminate feces (i.e., defecation dysfunction) can be a life-threatening condition. The current standard of care for defecation dysfunction includes digital extraction of feces from the rectum in combination with laxatives and a diet intended to soften the stool and promote its passage. Some individuals require large volume (1 L) warm water enemas that have a latency of 30 minutes to an hour before the water and fecal contents are expelled.

Individuals with defecation dysfunction may also experience episodes of incontinence or involuntary leakage of feces from the rectum. Regular emptying of the rectum by voluntary defecation or by use of a therapeutic agent that induces defecation reduces the chance of an incontinence episode thereafter.

Defecation dysfunction is extremely prevalent in patients with spinal cord injury, spina bifida, multiple sclerosis, Parkinson's disease, stroke, and other conditions involving spinal cord or other central nervous system pathology. Defecation dysfunction is also prevalent in subjects with Chronic Idiopathic Constipation (CIC) and constipation-predominant Irritable Bowel Syndrome (IBS-c). Defecation dysfunction is also common in elderly subjects, where it is a leading cause of institutionalization.

Spinal cord injury (SCI) is the most common injury that profoundly affects defecation and usually results from physical trauma (traffic accidents, sports injuries, armed combat) and also from infections, vascular disorders, cancers, congenital malformations, polio, tuberculosis, and other causes. It is estimated that the annual incidence of SCI, not including those who die at the scene of the injury, is approximately 54 cases per million population in the U.S. or approximately 17,730 new cases each year. The number of people in the U.S. who were alive in 2019 who had SCI was estimated to be approximately 291,000 persons.

Injury to the spinal cord and/or brain can lead to an inability to voluntarily defecate. Thus, the vast majority of individuals with SCI must invest considerable time in a “bowel program,” using digital rectal stimulation and manual extraction of stool, applied by themselves (56%) or by their caregivers (44%). The defecation reflex is triggered by inserting the fingers into the rectum and stroking the epithelial lining. This stroking imitates the physiological stimulation of rectal mechanoreceptors when stool passes into the rectum and activates the mechanoreceptors located in the mucosal layer of the rectum. Mechanoreceptor stimulation activates peripheral and spinal defecation reflexes that produce peristaltic contractions of the descending and sigmoid colon. These rectal contractions are accompanied by coordinated relaxation of the anal sphincters to allow passage of stool through the anal canal. Furthermore, mechanical stimulation stimulates the secretion of mucus and electrolytes which facilitate the passage of stool.

Bowel programs are onerous and consume anywhere from 30 minutes-2 hours in 56% of people with SCI, many of whom must perform their bowel program daily (42%) or every other day (23%). In addition to manual extraction of feces, large volume (1 L) warm water enemas are used that require sitting on the toilet for 30 minutes to an hour until the water and fecal contents are expelled. In some cases, a stimulant laxative is administered orally or intrarectally, although effects may last hours longer than necessary, and they cannot be administered chronically. These methods are degrading and can be personally demoralizing, altering social relationships, leading to depression, anger, poor self-image, embarrassment, frustration, and reduced productivity.

Fecal incontinence involves passing of solid or liquid stool or mucus from the rectum, which must first be present in the rectum. Removal of solid or liquid stool or mucus from the rectum by voluntary defecation or by use of a pharmacological agent that induces defecation reduces the chance of an incontinence episode for a short time afterwards.

Individuals with defecation dysfunction may also experience episodes of constipation, whereby individuals experience difficulty evacuating feces from the distal colon and rectum. Constipation is one of the most common gastrointestinal complaints in the United States. More than 40 million Americans have frequent constipation, accounting for 8 million physician visits a year. Self-treatment of constipation with over-the-counter (OTC) laxatives is by far the most common form of treatment. Around $2.5 billion is spent on OTC laxative products each year in America.

Constipation is common in a number of gastrointestinal tract disorders including but not limited to CIC, IBS-c, celiac disease or gluten-sensitive enteropathy, megacolon associated with hypothyroidism, pseudo-obstruction of the gastrointestinal tract, colitis, hypomotility of the colon associated with diabetes mellitus, adult onset Hirschsprung's disease, neurological disorders, myopathic disorders, spinal cord injury, multiple sclerosis, Parkinson's disease, stroke, spina bifida, jejunal-ileal bypass with secondary megacolon, cancer chemotherapy, critical illness including severe burns and other major stresses, major depression, the post-operative state, drug-induced constipation (e.g. opioids, antimuscarinics), and other pathological conditions.

Present treatments include OTC laxatives, AMITIZA (lubiprostone which is a chloride channel activator; approved for IBS-C and CIC), LINZESS (linaclotide; a guanylate cyclase-C agonist; approved for the treatment of IBS-C and CIC). However, none of these available medications produce colorectal contraction and expulsion of feces, rather they increase the fluid in the intestines to soften the stool and promote its passage. Their onset of action is delayed (typically several hours), which may increase the risk of incontinence. Due to the unpredictable timing of their actions, existing therapeutic agents cannot restore voluntary control over defecation.

Because existing therapies and treatments for defecation dysfunction are associated with limitations as described above, new therapies and treatments are desirable. The presently disclosed subject matter provides such new therapies and treatments to address these limitations.

### SUMMARY

In one embodiment of the presently disclosed subject matter, a method is provided for treating defecation dysfunction in a mammal in need of treatment, which comprises administering on an as-needed basis a therapeutically effective amount of a transient receptor potential vanilloid 1 (TRPV1) receptor agonist or a pharmaceutically acceptable salt thereof, wherein the TRPV1 receptor agonist or the pharmaceutically acceptable salt thereof, has a rapid onset and a short duration of action, to induce the defecation.

In one embodiment of the presently disclosed subject matter, a method is provided for treating defecation dysfunction in a mammal in need of treatment, which comprises administering on an as-needed basis to the mammal a therapeutically effective amount of a TRPV1 receptor agonist, or a pharmaceutically acceptable salt thereof, to induce the defecation.

In one embodiment of the presently disclosed subject matter, a pharmaceutical formulation is provided for treating defecation dysfunction in a mammal in need of treatment on an as-needed basis, which comprises a therapeutically effective amount of a rapid onset and short acting TRPV1 receptor agonist, or a pharmaceutically acceptable salt thereof, and a carrier for administration of the TRPV1 receptor agonist to the mammal on the as-needed basis.

In one embodiment of the presently disclosed subject matter, a pharmaceutical formulation is provided for treating defecation dysfunction in a mammal in need of treatment on an as-needed basis, which comprises a therapeutically effective amount of a TRPV1 receptor agonist, or a pharmaceutically acceptable salt thereof, and a carrier for administration of a TRPV1 receptor agonist to the mammal on the as-needed basis.

In one embodiment of the presently disclosed subject matter, a packaged kit is provided for a patient to use in the treatment of defecation dysfunction, comprising a pharmaceutical formulation of a therapeutically effective amount of a rapid onset and short acting TRPV1 receptor agonist, or a pharmaceutically acceptable salt thereof; a container housing the pharmaceutical formulation during storage and prior to administration; and instructions for application.

In one embodiment of the presently disclosed subject matter, a packaged kit is provided for a patient to use in the treatment of loss of or decrease in voluntary control of defecation, comprising a pharmaceutical formulation of a therapeutically effective amount of a TRPV1 receptor agonist, or a pharmaceutically acceptable salt thereof; a container housing the pharmaceutical formulation during storage and prior to administration; and instructions for carrying out administration in a manner effective to treat the loss or decrease in control.

In one embodiment of the presently disclosed subject matter, a packaged kit is provided for a patient to use for treating defecation dysfunction on an as-needed basis, comprising a pharmaceutical formulation of a therapeutically effective amount of a rapid onset and short acting TRPV1 receptor agonist, or a pharmaceutically acceptable salt thereof; a container housing the pharmaceutical formulation during storage and prior to administration; and instructions for carrying out administration on the as-needed basis to treat the defecation dysfunction.

In one embodiment of the presently disclosed subject matter, a packaged kit is provided for a patient to use for treating defecation dysfunction on an as-needed basis, comprising a pharmaceutical formulation of a therapeutically effective amount of a TRPV1 receptor agonist, or a pharmaceutically acceptable salt thereof; a container housing the pharmaceutical formulation during storage and prior to administration; and instructions for carrying out administration on the as-needed basis to treat the defecation dysfunction.

In one embodiment of the presently disclosed subject matter, a method is provided for inducing defecation in a mammal, which comprises administering on an as-needed basis to the mammal a therapeutically effective amount of a TRPV1 receptor agonist or a pharmaceutically acceptable salt thereof, wherein the TRPV1 receptor agonist or the pharmaceutically acceptable salt thereof, has a rapid onset and a short duration of action, to induce defecation.

In one embodiment of the presently disclosed subject matter, a method is provided for inducing defecation in a mammal, which comprises administering on an as-needed basis to the mammal a therapeutically effective amount of a TRPV1 receptor agonist, or a pharmaceutically acceptable salt thereof, to induce defecation.

### BRIEF DESCRIPTION OF THE FIGURES

The foregoing aspects and other features of the invention are explained in the following description, taken in connection with the accompanying drawings.

FIG. 1 is a schematic diagram showing how rectal afferent stimulation initiates defecation.

FIG. 2 is a physiograph tracing that shows intrarectal nonivamide, a TRPV1 receptor agonist, produced a rapid, transient increase in rectal pressure.

FIG. 3 is a physiograph tracing that shows intrarectal nonivamide, a TRPV1 receptor agonist, produced repeatable increases in rectal pressure.

FIG. 4 is a physiograph tracing that shows intrarectal nonivamide, a TRPV1 receptor agonist, produced increased rectal pressure that was sufficient to expel a balloon catheter.

FIG. 5 is a physiograph tracing that shows intrarectal OEA, a TRPV1 receptor agonist, produced a rapid, transient increase in rectal pressure.

FIG. 6 is a physiograph tracing that shows intrarectal OLDA, a TRPV1 receptor agonist, produced a rapid, transient increase in rectal pressure.

FIGS. 7A, 7B, and 7C are figures in association with Example 7. FIG. 7A is a physiograph tracing and FIGS. 7B and 7C are bar graphs that show capsaicin, a TRPV1 receptor agonist, produced a rapid, transient increase in rectal pressure in anesthetized rats, equal to pressures produced by other TRPV1 receptor agonists, and is sufficient to produce defecation measured as number of fecal pellets and weight of feces in the awake rat.

FIG. 8 is a physiograph tracing that shows intrarectal capsaicin produced a transient, repeatable increase in rectal pressure.

FIG. 9 is a set of physiograph tracings that show intrarectal administration of a capsaicin-soaked cotton ball suppository produced rectal pressure responses similar to those produced by intrarectal capsaicin solution dosing.

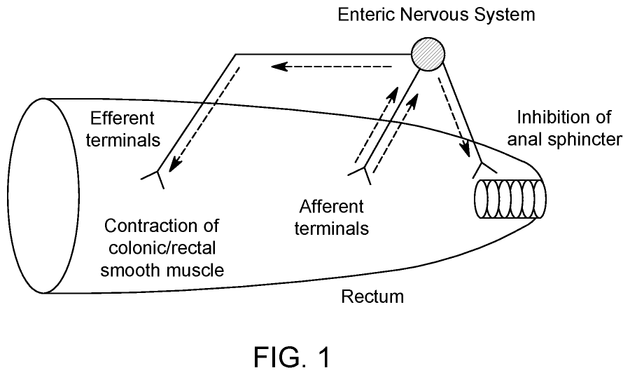

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Described herein is a method for inducing defecation in a mammal. The method comprises administering to the mammal a therapeutically effective amount of transient receptor potential vanilloid 1 (TRPV1) receptor agonist or a pharmaceutically acceptable salt thereof. Administration is by an intrarectal mode. The TRPV1 receptor agonist or the pharmaceutically acceptable salt thereof, induces defecation.

Also described is a method for treating defecation dysfunction in a mammal in need of treatment. The method comprises administering to the mammal a therapeutically effective amount of a transient receptor potential vanilloid 1 (TRPV1) receptor agonist or a pharmaceutically acceptable salt thereof. Administering is by an intrarectal mode, and the TRPV1 receptor agonist or the pharmaceutically acceptable salt thereof induces defecation.

In one embodiment of the presently disclosed subject matter, a method is provided for treating defecation dysfunction in a mammal in need of treatment, which comprises administering on an as-needed basis to the mammal a therapeutically effective amount of a transient receptor potential vanilloid 1 (TRPV1) agonist or a pharmaceutically acceptable salt thereof, wherein the TRPV1 receptor agonist or the pharmaceutically acceptable salt thereof has a rapid onset and a short duration of action, to induce defecation. The compositions and methods of the present disclosure meet an existing need for new treatments for defecation dysfunction including, for example, constipation and the inability to voluntarily defecate. The defecation dysfunction can be a result of a wide range of injuries, conditions, diseases, or disorders, including of one or more of spinal cord injury, traumatic brain injury, multiple sclerosis, spina bifida, degenerative brain disease, Alzheimer's, Parkinson's, dementia, diabetes, advanced age, drug-induced, and postoperative status.

In one embodiment of the presently disclosed subject matter, a method is provided for inducing defecation in a mammal, which comprises administering on an as-needed basis to the mammal a therapeutically effective amount of a TRPV1 receptor agonist or a pharmaceutically acceptable salt thereof, wherein the TRPV1 receptor agonist or the pharmaceutically acceptable salt thereof, has a rapid onset and a short duration of action, to induce defecation. The compositions and methods of the present disclosure meet an existing need for new treatments to induce defecation in persons who are, for example, unconscious to cause the defecation before the person defecates unconsciously. Another advantage of the methods and compositions of the present disclosure is for a mammal in need or a pet owner who may want to induce defecation in their normal dog, for example, at a specific, convenient location or time.

To provide an effective treatment for anal sphincter dyssynergia, the administering of the TRPV1 receptor agonist according to the methods and formulations of the present disclosure may be combined with one or more sphincter relaxants such as, but not limited to, alpha adrenergic receptor blockers, nitric oxide (NO) donors, PDE5 inhibitors, and prostaglandin E receptor (EP1,2,3) agonists.

A therapeutically effective amount of one or more TRPV1 receptor agonists may be administered via intrarectal administration.

Methods are provided herein for using an active agent or otherwise referred to herein interchangeably as a “pharmaceutical agent” to provide drug-induced defecation. The drug-induced defecation can be one or more of on demand, rapid onset, and/or short duration. The drug-induced defecation can be useful for those with defecation dysfunction or for a mammal for which inducing defecation is otherwise desirable. The active agents or pharmaceutical agents of the present disclosure can include TRPV1 receptor agonists. While there are known pharmaceutical agents that are TRPV1 receptor agonists, systemic administration of these agents to a living being can produce toxicity as opposed to therapeutic defecation. In contrast, the compositions and methods of the present disclosure provide pharmaceutical formulations and methods of administration of TRPV1 receptor agonists providing a duration of action which can produce defecation and then allow the rectum to subsequently relax to allow for storage of newly-formed stool to prevent subsequent incontinence or other deleterious effects. The formulations and methods of administration of the present disclosure can minimize the duration of side-effects.

One advantage of the presently described subject matter is provision of TRPV1 receptor agonists that have a rapid-onset and short duration of action for administration to mammals to achieve a rapid-onset and short duration contraction of the rectum. Surprisingly, the contractions produced by administration of these TRPV1 receptor agonists can actually elicit physiologically significant contraction of the rectum, anal sphincter relaxation, and defecation. This is unexpected since stimulation of TRPV1 receptors in the rectum might have caused contraction and closure of the anal sphincter and thus prevention of defecation.

Another advantage of the presently described subject matter is that the TRPV1 receptor agonist-induced defecation can be achieved without the adverse effect of oral burning sensations and contractions of the entire GI tract to produce painful cramps.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

By “SCI” is meant spinal cord injury.

By “GI” is meant gastrointestinal tract.

By “TRPV1” is meant transient receptor potential vanilloid 1 (TRPV1). Exemplary TRPV1 receptor agonists include, without limitation, capsaicin, resiniferatoxin, nonivamide, anandamide, olvanil, palvanil, arvanil, piperine, N-Oleoyldopamine, and SDZ-249665.

By an “effective” amount or a “therapeutically effective amount” of a drug or pharmacologically active agent of the present disclosure including, for example, a TRPV1 receptor agonist, or a pharmaceutically acceptable salt thereof, is meant a nontoxic but sufficient amount of the drug or active agent to provide the desired effect, i.e., treating defecation dysfunction such as effectuating voluntary defecation. It is recognized that the effective amount of a drug or pharmacologically active agent will vary depending on the means of administration, the selected compound, and the species to which the drug or pharmacologically active agent is administered. It is also recognized that one of skill in the art will determine appropriate effective amounts by taking into account such factors as metabolism, bioavailability, and other factors that affect levels of a drug or pharmacologically active agent following administration within the unit dose ranges disclosed further herein.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”) as in a “pharmacologically active” derivative or metabolite, refers to a derivative or metabolite having the same type of pharmacological activity as the parent compound. When the term “pharmaceutically acceptable” is used to refer to a derivative (e.g., a salt or an analog) of an active agent, it is to be understood that the compound is pharmacologically active as well, i.e., therapeutically effective for treating defecation dysfunction.

By “continuous” dosing is meant the chronic administration of a selected active agent.

By “as-needed” dosing, also known as “pro re nata” or “prn” dosing, and “on demand” dosing or administration is meant the administration of a single dose of the active agent at some time prior to commencement of defecation. Administration can be immediately prior to such a time, including about 0 minutes, about 0 to about 5 minutes, about 0 to about 10 minutes, about 0 to about 20 minutes, about 0 to about 30 minutes, or about 0 to about 40 minutes, prior to such a time, depending on the formulation and the route of administration.

By “rapid-onset” is intended any period of time up to and including a drug onset between about 0 sec to about 1 hour, between about 0 sec to about 45 minutes, between about 0 sec to about 30 minutes, between about 0 sec to about 15 minutes, or between about 0 sec to about 10 minutes, or between 0 sec to 5 min, after active agent administration.

By “short duration of action” is intended duration between about 2 hours to about 10 minutes, between about 1 hour to about 10 minutes, and between about 30 minutes to about 10 minutes, and between 15 to about 5 minutes after active agent administration.

The term “immediate release” is used in its conventional sense to refer to a drug formulation that provides for release of the drug immediately after drug administration.

The term “defecation dysfunction” refers to the inability to expel feces from the distal bowel and rectum. Defecation dysfunction is also known as defecation disorder, anorectal evacuation disorder, fecal impaction, constipation, or colonic dysmotility.

“Rectal” or “intrarectal” drug delivery, refers to delivery of a drug into an individual's rectum.

The defecation reflex is triggered by physiological stimulation of rectal mechanoreceptors when stool passes into the rectum and activates mechanoreceptors located in the rectum. Mechanoreceptor stimulation activates peripheral and spinal defecation reflexes that produce peristaltic contractions of the descending and sigmoid colon. These rectal contractions are accompanied by coordinated relaxation of the anal sphincters to allow passage of stool through the anal canal (MacDonagh, Sun et al. 1992, Shafik 1996, Shafik 1999, Shafik, El-Sibai et al. 2001, Callaghan, Furness et al. 2018). Furthermore, mechanical stimulation causes secretion of mucus and electrolytes, which facilitates the passage of stool. It can take an hour or more of digital stimulation applied during the bowel program to produce colonic contractions capable of inducing defecation.

Intrarectal and oral stimulant laxatives (e.g., bisacodyl or senna) have been used to “sensitize” rectal mechanoreceptors and chemoreceptors to facilitate defecation. These stimulants permeabilize the mucosal barrier of the rectum, allowing irritative substances in the feces to activate nociceptive afferent nerve terminals in the submucosa. Afferent activation signals the GI tract to secrete mucus and electrolytes, contract the rectum, relax the anal sphincter, and eliminate the irritating substances. However, these agents can require 30 min to several hours initiate defecation, and they can continue to stimulate defecation for hours after administration, which raises concern about post-defecation fecal incontinence. They also cannot be used every day due to long-term damage of the lining of the GI tract and tolerance to the laxative effects.

The TRPV1 receptor functions as a non-selective cation channel and polymodal receptor that is formed by a tetrameric complex with each subunit containing six N-terminal ankyrin repeats. TRPV1 receptors are primarily expressed on sensory nerves, and activation by agonists modulates the activity of sensory nerves. For example, activation of TRPV1 receptors on afferent pain-sensing nerve fibers mediates the painful sensation of capsaicin that may be experienced following ingestion of chili peppers.

In the GI tract, TRPV1 receptors are abundantly expressed in nerve fibers of the distal colon and rectum of the mouse, where they mediate capsaicin-induced contractions of distal colon and rectum (Matsumoto, Kurosawa et al. 2009). Capsaicin has been used as a pharmacological tool to induce overactivity of the colon and initiate defecation when administered intracolonically in conscious dogs (Hayashi, Shibata et al. 2010, Kikuchi, Shibata et al. 2014); the effect of intrarectal application in dogs has not been reported previously. In humans, activation of rectal afferent terminals by capsaicin has been established in subjects who reported discomfort and “an urge to defecate”, without reported defecation, following intrarectal administration of capsaicin (van Wanrooij, Wouters et al. 2014).

No sensations of discomfort would be expected from intrarectal administration of TRPV1 receptor agonists in individuals with complete spinal cord injury (SCI) due to complete disruption of spinal sensory pathways. In individuals who experience rectal sensations, it is possible that mild rectal discomfort would be preferable to the physical discomfort, stigma, time, and effort required for digital stimulation and manual extraction of stool currently experienced by individuals with incomplete SCI.

Intrarectal administration offers a safety advantage because it concentrates exposure at the target tissue. As the desired therapeutic effects of intrarectal TRPV1 receptor agonists are expulsion of the rectal contents and secretion of electrolytes, fluids, and mucus, it is expected that the TRPV1 receptor agonist will be rapidly eliminated from the rectum, terminating its actions and avoiding significant systemic absorption. Furthermore, rectal absorption of TRPV1 receptor agonists may be less extensive than oral absorption.

TRPV1 receptor agonists and their structures are listed in Table 1.

Capsaicinoids are naturally occurring TRPV1 receptor agonists that constitute a family of similar alkaloid molecules isolated from plants of the Capsicum genus (Fattori, Hohmann et al. 2016) and are amides formed from condensation of vanillylamine and fatty acids of different chain lengths (Reyes-Escogido Mde, Gonzalez-Mondragon et al. 2011). Capsaicinoids include capsaicin, dihydrocapsaicin, nordihydrocapsaicin, homodihydrocapsaicin, and homocapsaicin, among others, all of which are structural analogs. Resiniferatoxin is an example of a naturally occurring resiniferoid isolated from the Euphorbia resinifera plant. Resiniferatoxin is an extremely potent TRPV1 receptor agonist.

Other TRPV1 receptor agonists include: olvanil, palvanil, arvanil, piperine, N-Oleoyldopamine, and SDZ-249665, among others (Table 1).

TABLE 1
TRPV1 Receptor Agonists

| | |
|---|---|
| AM 404 | 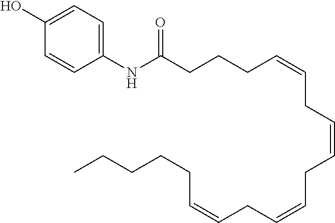 [SMILES: CCCCC/C=C\C/C=C\C/C=C\C/C=C\CCCC(=O)Nc1ccc(O)cc1] |
| Anandamide | 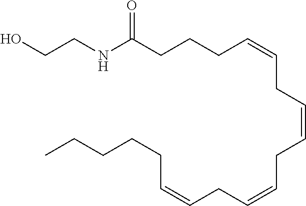 [SMILES: CCCCC/C=C\C/C=C\C/C=C\C/C=C\CCCC(=O)NCCO] |
| Arvanil | 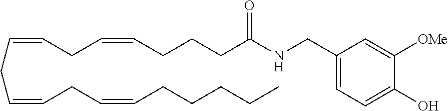 [SMILES: CCCCC/C=C\C/C=C\C/C=C\C/C=C\CCCC(=O)NCc1ccc(O)c(OC)c1] |
| Capsaicin | 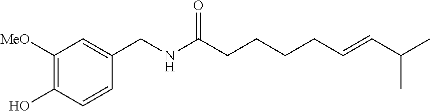 [SMILES: COc1cc(CNC(=O)CCCC/C=C/C(C)C)ccc1O] |
| GSK 1016790A | 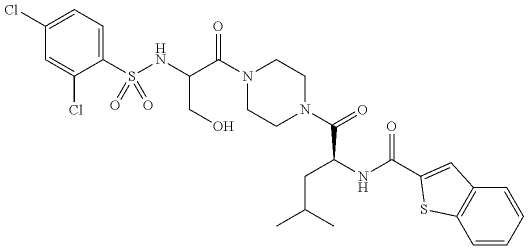 [SMILES: CC(C)C[C@H](NC(=O)c1cc2ccccc2s1)C(=O)N1CCN(C(=O)C(CO)NS(=O)(=O)c2ccc(Cl)cc2Cl)CC1] |
| NADA N-Arachidonoyl dopamine | 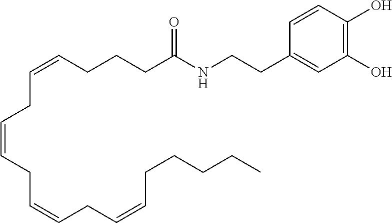 [SMILES: CCCCC/C=C\C/C=C\C/C=C\C/C=C\CCCC(=O)NCCc1ccc(O)c(O)c1] |
| OLDA N-Oleoyldopamine | 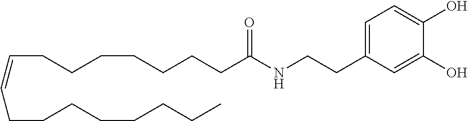 [SMILES: CCCCCCCC/C=C\CCCCCCCC(=O)NCCc1ccc(O)c(O)c1] |
| Olvanil | 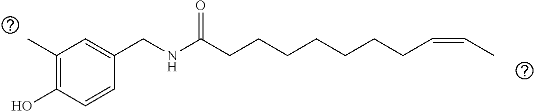 [SMILES: C/C=C\CCCCCCCC(=O)NCc1ccc(O)c(C)c1] |
| PPAHV Phorbol 12-phenylacetate 13-acetate 20- homovanillate | 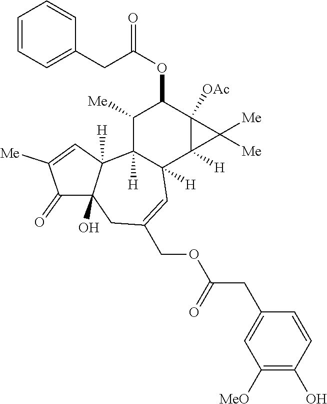 |
| OEA Oleoylethanolamide | 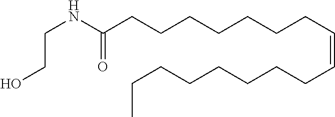 [SMILES: CCCCCCCC/C=C\CCCCCCCC(=O)NCCO] |
| Nonivamide | 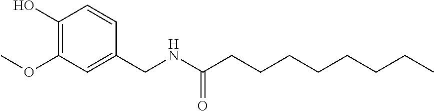 [SMILES: CCCCCCCCC(=O)NCc1ccc(O)c(OC)c1] |
| Palvanil | 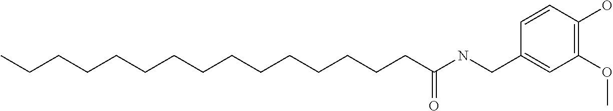 [SMILES: CCCCCCCCCCCCCCCC(=O)NCc1ccc(O)c(OC)c1] |
| (E)-Capsaicin | 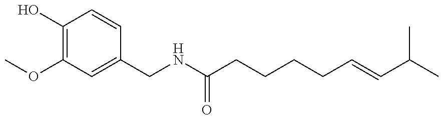 [SMILES: COc1cc(CNC(=O)CCCC/C=C/C(C)C)ccc1O] |
| Piperine | 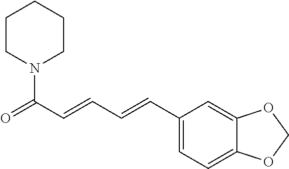 [SMILES: O=C(/C=C/C=C/c1ccc2c(c1)OCO2)N1CCCCC1] |
| N-Oleoyldopamine | 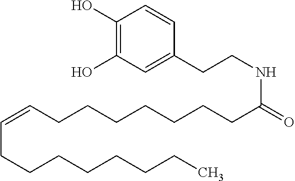 [SMILES: CCCCCCCC/C=C\CCCCCCCC(=O)NCCc1ccc(O)c(O)c1] |
| Dihydrocapsaicin | 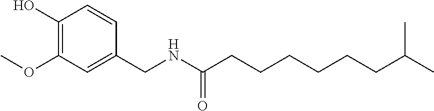 [SMILES: COc1cc(CNC(=O)CCCCCCC(C)C)ccc1O] |
| Homodihydrocapsaicin | 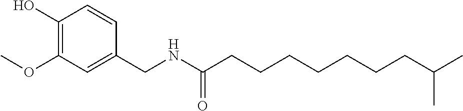 [SMILES: COc1cc(CNC(=O)CCCCCCCC(C)C)ccc1O] |
| Nordihydrocapsaicin | 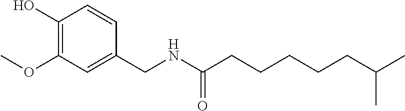 [SMILES: COc1cc(CNC(=O)CCCCCC(C)C)ccc1O] |
| Homocapsaicin | 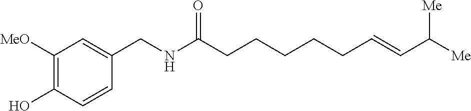 [SMILES: COc1cc(CNC(=O)CCCCC/C=C/C(C)C)ccc1O] |

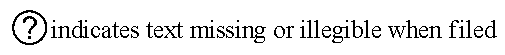

In one embodiment, the presently disclosed subject matter provides a strategy for administering pharmacological active agents, compositions, and formulations that have distinct pharmacokinetic properties of a rapid-onset of action and a short duration of action, as well as pharmacodynamic properties to contract the distal colon and rectum and induce defecation “on demand” when convenient and appropriate for a person suffering from a defecation dysfunction or otherwise in need of such treatment such as, for example, a person who is unconscious and may defecate unconsciously. The compositions and methods of the present disclosure may also be useful for a mammal in need or to allow a pet owner to induce defecation in their pet at a specific, convenient location or time.

In embodiments, increases in rectal pressure induced by TRPV1 receptor agonists are sufficient to induce defecation. Rectal pressures over 20 mmHg have been shown to be sufficient to induce defecation in anesthetized rats and awake rats and in decerebrate rats. Example 7 below illustrates that capsaicin, a TRPV1 receptor agonist, produced a rapid, transient increase in rectal pressure in anesthetized rats, equal to pressures produced by other TRPV1 receptor agonists, which was sufficient to produce defecation measured as number of fecal pellets and weight of feces in the awake rat. Examples 4, 5, and 6 below illustrate that other TRPV1 receptor agonists, including nonivamide, OEA and OLDA, induce pressures meeting or exceeding those induced by capsaicin in Example 7. Accordingly, a person having ordinary skill in the art would expect nonivamide, OEA, OLDA, and other similar TRPV1 agonists to induce defecation in the same way that capsaicin did.

In one embodiment of the present disclosure, a method is provided for treating defecation dysfunction in a mammal in need of treatment. In one embodiment of the present disclosure, a method is provided for inducing defecation in a mammal that may not necessarily have a defecation dysfunction. The methods include administering on an as-needed basis to the mammal a therapeutically effective amount of a TRPV1 receptor agonist or a pharmaceutically acceptable salt thereof, wherein the TRPV1 receptor agonist or the pharmaceutically acceptable salt thereof, has a rapid onset and a short duration of action. The TRPV1 receptor agonist can include, but is not limited to, resiniferatoxin. dihydrocapsaicin, nordihydrocapsaicin, homodihydrocapsaicin, homocapsaicin, olvanil, palvanil, arvanil, piperine, nonivamide, N-Oleoyldopamine, SDZ-249665 and anandamide and others listed in Table 1. The mammal can include but is not limited to, for example, a human, a cat, or a dog.

Formulations of the compositions and active agents of the present disclosure are provided in as-needed dosage forms, and can include short-term, rapid-onset, rapid-offset, controlled release, delayed release, and pulsatile release formulations, so long as they are formulated to achieve as-needed administration of an active agent, as defined further herein.

In all of the methods and compositions of the present disclosure, the rapid-onset of the TRPV1 receptor agonist can be characterized by an onset of action ranging from about 0 sec to about 1 hour after TRPV1 receptor agonist administration, ranging from about 0 sec to about 45 minutes after TRPV1 receptor agonist administration, ranging from about 0 sec to about 30 minutes after TRPV1 receptor agonist administration, ranging from about 0 sec to about 15 minutes after TRPV1 receptor agonist administration, ranging from about 0 sec to about 10 minutes after TRPV1 receptor agonist administration, or ranging from about 0 sec to about 5 min after TRPV1 receptor agonist administration.

In all of the methods and compositions of the present disclosure, the short duration of action of the TRPV1 receptor agonist can be characterized by a duration of action ranging from about 1 hour to about 10 minutes after TRPV1 receptor agonist administration, ranging from about 30 minutes to about 10 minutes after TRPV1 receptor agonist administration, or ranging from about 15 to about 1 minute after TRPV1 receptor agonist administration.

The TRPV1 receptor agonist, or the pharmaceutically acceptable salt thereof, can be formulated as an immediate release dosage form and the as-need administering can range from about 0 minutes to about 40 minutes prior to when the defecation is desired, from about 0 minutes to about 20 minutes prior to when the defecation is desired, or from about 0 minutes to about 5 minutes prior to when the defecation is desired.

In one embodiment, one or more additional active agents can be administered either simultaneously or sequentially with the TRPV1 receptor agonist active agent in either a separate or a single formulation. The additional active agent may be one that is effective in treating bowel dysfunction that accompanies fecal retention. The additional active agent may be one that potentiates the effect of a TRPV1 receptor agonist active agent for treating defecation dysfunction. Suitable additional active agents include, but are not limited to, for example, laxatives, alpha adrenergic antagonists (e.g., silodosin, terazosin, tamsulosin, doxazosin, prazosin, alfuzosin), phosphodiesterase inhibitors (e.g., sildenafil, vardenafil, tadalafil) lubiprostone, linaclotide, and/or any agent that does not inhibit the action of the primary active agent.

The additional active agent may be a compound that can induce one of colon contraction and/or anal sphincter relaxation in the subject. The anal sphincter relaxant agent can be, for example, one of vasoactive intestinal polypeptide (VIP), a NO donor, amyl nitrate, butyl nitrate, glyceryl trinitrate, an alpha-adrenergic receptor blocker, tamsulosin, silodosin, alfuzosin, or naftopidil or other suitable anal sphincter relaxant agents.

Any of the active agents may be administered in the form of a salt, ester, amide, prodrug, active metabolite, derivative, or the like, provided that the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts are prepared from the free base using conventional methodology and involves reaction with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are salts prepared with organic acids. Conversely, preparation of basic salts of acid moieties which may be present on an active agent are prepared in a similar manner using a pharmaceutically acceptable base such as sodium hydroxide, potassium hydroxide, ammonium hydroxide, calcium hydroxide, trimethylamine, or the like.

Preparation of esters involves functionalization of hydroxyl and/or carboxyl groups that may be present within the molecular structure of the drug. The esters are typically acyl-substituted derivatives of free alcohol groups, i.e., moieties that are derived from carboxylic acids of the formula RCOOH where R is alkyl, and preferably is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety, which results in a compound that is therapeutically inactive until modified by an individual's metabolic system.

Other salts, enantiomers, analogs, esters, amides, prodrugs, active metabolites, and derivatives of the active agents may be prepared using standard techniques known to those skilled in the art of synthetic organic chemistry or may be deduced by reference to the pertinent literature. In addition, chiral active agents may be in isomerically pure form, or they may be administered as a racemic mixture of isomers.

The active agents of the present disclosure can be administered by a mode including intrarectal administration.

The active agents of the present disclosure can be contained within a pharmaceutical formulation. The pharmaceutical formulation can be a unit dosage form. The pharmaceutical formulation can be selected from the group consisting of suppositories, capsules, tablets, powders, creams, ointments, gels, foams, solutions, emulsions, and suspensions.

The pharmaceutical formulation can be selected from the group consisting of suppositories, creams, ointments, gels, foams, solutions, emulsions, and suspensions. The pharmaceutical formulation can include a permeation enhancer.

Suitable compositions and dosage forms are suppositories, capsules, tablets, powders, creams, ointments, gels, foams, solutions, emulsions, and suspensions for rectal administration, Further, those of ordinary skill in the art can readily deduce that suitable formulations involving these compositions and dosage forms, including those formulations as described elsewhere herein.

Tablets may be manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

In addition to the active agent(s), tablets prepared for intrarectal administration using the method of the disclosure will generally contain other materials such as binders, diluents, lubricants, disintegrants, fillers, stabilizers, surfactants, preservatives, coloring agents, flavoring agents and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum. Diluents are typically necessary to increase bulk so that a practical size tablet is ultimately provided. Suitable diluents include dicalcium phosphate, calcium sulfate, lactose, cellulose, kaolin, mannitol, sodium chloride, dry starch and powdered sugar. Lubricants are used to facilitate tablet manufacture; examples of suitable lubricants include, for example, vegetable oils such as peanut oil, cottonseed oil, sesame oil, olive oil, corn oil, and oil of theobroma, glycerin, magnesium stearate, calcium stearate, and stearic acid. Stearates, if present, preferably represent at no more than approximately 2 wt. % of the drug-containing core. Disintegrants are used to facilitate disintegration of the tablet, and are generally starches, clays, celluloses, algins, gums or crosslinked polymers. Fillers include, for example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. Stabilizers are used to inhibit or retard drug decomposition reactions that include, by way of example, oxidative reactions. Surfactants may be anionic, cationic, amphoteric or nonionic surface active agents.

The dosage form may also be a capsule, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. (See, for e.g., Ansel's Pharmaceutical Dosage Forms and Drug Delivery Systems 9th Edition by Loyd V. Allen, Nicholas G. Popovich, Howard C. Ansel 2011), which describes materials and methods for preparing encapsulated pharmaceuticals. If the active agent-containing composition is present within the capsule in liquid form, a liquid carrier is necessary to dissolve the active agent(s). The carrier must be compatible with the capsule material and all components of the pharmaceutical composition and must be suitable for intrarectal insertion.

The dosage form may also be a capsule, comprising a hard or soft capsule containing a fill consisting of one or more inert ingredients, and one or more coatings on the capsule, wherein at least one coating comprises a suitable formulation and dosage form.

Preferred intrarectal dosage forms include rectal suppositories, capsules, tablets, powders, creams, ointments, gels, foams, solutions, emulsions, and suspensions (Jannin, Lemagnen et al. 2014). The suppository, capsule, tablet, powder, cream, ointment, gel, foam, solution, emulsion, or suspension formulation for intrarectal delivery comprises a therapeutically effective amount of the selected active ingredient and one or more conventional nontoxic carriers suitable for intrarectal drug administration. The intrarectal dosage forms of the present invention can be manufactured using conventional processes. The intrarectal dosage unit can be fabricated to disintegrate rapidly or over a period of several hours. The time period for complete disintegration is preferably in the range of from about 1 minute to about 6 hours, and optimally is less than about 3 hours.

For intrarectal administration, the formulation comprises a rectal dosage form containing the active agent and one or more selected carriers or excipients, such as water, silicone, waxes, petroleum jelly, polyethylene glycol (“PEG”), propylene glycol (“PG”), liposomes, sugars such as mannitol and lactose, and/or a variety of other materials, with polyethylene glycol and derivatives thereof particularly preferred.

Intrarectal drug administration can be carried out in a number of different ways using a variety of rectal dosage forms. For example, the drug can be introduced into the rectum from a flexible tube, squeeze bottle, or pump. The drug may also be contained in coatings, pellets, tablets, capsules or suppositories that are absorbed, melted or bioeroded in the rectum. In certain embodiments, the drug is included in a coating on the exterior surface of a rectal insert. It is preferred, although not essential, that the drug be delivered from at least about 1 cm into the rectum, and preferably from at least about 4 cm into the rectum. Generally, delivery from at least about 3 cm to about 8 cm into the rectum will provide effective results in conjunction with the present method.

Rectal suppository formulations containing PEG, hard fats, glycerol, gelatins or polyglycerol esters may be conveniently formulated using conventional techniques, e.g., compression molding, heat molding or the like, as will be appreciated by those skilled in the art and as described in the pertinent literature and pharmaceutical texts. (See, e.g., Ansel's Pharmaceutical Dosage Forms and Drug Delivery Systems 9th Edition by Loyd V. Allen, Nicholas G. Popovich, Howard C. Ansel 2011), which discloses typical methods of preparing pharmaceutical compositions in the form of rectal suppositories. The PEG or PEG derivative preferably has a molecular weight in the range of from about 200 to about 3500 g/mol, more preferably in the range of from about 1,000 to about 2,000 g/mol. Suitable polyethylene glycol derivatives include polyethylene glycol fatty acid esters, for example, polyethylene glycol monostearate, polyethylene glycol sorbitan esters, e.g., polysorbates, and the like. Hard fats, oils etc. (e.g., cocoa butter, Wipetsol^{R}) can also be used. Depending on the particular active agent, it may also be preferred that rectal suppositories contain one or more solubilizing agents effective to increase the solubility of the active agent in the PEG or other intrarectal vehicle.

The rectal dosage form will preferably comprise a suppository that is on the order of from about 0.1 to about 10 cm in length, preferably from about 0.5 to about 3 cm in length, and less than about 2 cm in width, preferably less than about 1 cm in width. The weight of the suppository will typically be in the range of from about 0.5 gm to about 5 gm, preferably in the range of from about 1 gm to about 2 gm. However, it will be appreciated by those skilled in the art that the size of the suppository can and will vary, depending on the potency of the drug, the nature of the formulation, and other factors. Other components may also be incorporated into the intrarectal dosage forms described herein. The additional components include, but are not limited to, stiffening agents, antioxidants, preservatives, and the like. Examples of stiffening agents that may be used include, for example, paraffin, white wax and yellow wax. Preferred antioxidants, if used, include sodium bisulfite and sodium metabisulfite.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Ansel's Pharmaceutical Dosage Forms and Drug Delivery Systems 9th Edition by Loyd V. Allen, Nicholas G. Popovich, Howard C. Ansel 2011, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glycerylmonostearate, lanolin and stearic acid. Preferred water-soluble ointment bases are prepared from polyethylene glycols of varying molecular weight (See, e.g., Ansel's Pharmaceutical Dosage Forms and Drug Delivery Systems 9th Edition by Loyd V. Allen, Nicholas G. Popovich, Howard C. Ansel 2011).

Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the CARBOPOL trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Various additives, known to those skilled in the art, may be included in the intrarectal formulations. For example, solubilizers may be used to solubilize certain active agents. For those drugs having an unusually low rate of permeation through the mucosal tissue, it may be desirable to include a permeation enhancer in the formulation. Examples of suitable intrarectal permeation enhancers include dimethylsulfoxide (“DMSO”), dimethyl formamide (“DMF”), N, N-dimethylacetamide (“DMA”), decylmethylsulfoxide (“C10 MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark AZONE from Nelson Research & Development Co., Irvine, Calif.), SEPA (available from Macrochem Co., Lexington, Mass.), surfactants as discussed above, including, for example, TERGITOL, NONOXYNOL-9 and TWEEN-80, and lower alkanols such as ethanol.

One of skill in the art recognizes that the concentration of the active agent in any of the aforementioned dosage forms and compositions can vary a great deal and will depend on a variety of factors, including the type of composition or dosage form, the corresponding mode of administration, the nature and activity of the specific active agent, and the intended drug release profile. Preferred dosage forms contain a unit dose of active agent, i.e., a single therapeutically effective dose. For creams, ointments, etc., a “unit dose” requires an active agent concentration that provides a unit dose in a specified quantity of the formulation to be applied. The unit dose of any particular active agent will depend, of course, on the active agent and on the mode of administration. Similarly, the affinity of TRPV1 receptor agonists for the TRPV1 receptor is expected to differ substantially between each structurally distinct TRPV1 receptor agonist, and doses and concentrations of each TRPV1 receptor agonist should be adjusted based on comparison of each distinct TRPV1 receptor agonist's affinity for the TRPV1 receptor to the affinity of, for example, capsaicin for the TRPV1 receptor when deciding on an appropriate dose for each distinct TRPV1 receptor agonist.

TRPV1 receptor affinity is easily determined using standard receptor binding protocols, such as generating concentration displacement curves of radiolabeled capsaicin to establish Ki. Using similar if not identical binding conditions, the binding affinity of certain of the TRPV1 receptor agonists of the present disclosure including, for example, capsaicin can be established. If the TRPV1 receptor agonist of choice has a lower affinity than capsaicin for the TRPV1 receptor, then higher plasma concentrations would need to be achieved when dosing the particular TRPV1 receptor agonist than the concentrations observed for capsaicin. On the other hand, if the TRPV1 receptor agonist has a higher affinity than capsaicin for the TRPV1 receptor, then lower plasma concentrations can be used to achieve therapeutic benefit when dosing with the TRPV1 receptor agonist. For example, if the TRPV1 receptor agonist has an affinity 10× lower than capsaicin, then the plasma concentration to achieve a therapeutic benefit (e.g., nM) for the TRPV1 receptor agonist can be 10× higher than capsaicin.

For dosing of active agents exhibiting TRPV1 receptor agonist activity and having a rapid-onset and short duration of action, regardless of the formulation or mode of delivery, pharmacokinetic profiles (i.e., Cₘₐₓ, Tₘₐₓ, and T_{1/2}) can be provided as described herein to produce a therapeutic benefit, (e.g., in the range of about 1 ng/kg to about 100 mg/kg with compensation for TRPV1 receptor affinity).

For active agents exhibiting TRPV1 receptor agonist activity, including rapid-onset compounds exhibiting TRPV1 receptor agonist activity and/or short acting compounds exhibiting TRPV1 receptor agonist activity, the unit dose for intrarectal administration can be in the range of from about 1 ng to about 10,000 mg, in the range of from about 100 ng to about 5,000 mg. The unit dose for intrarectal administration can be greater than about 1 ng, about 5 ng, about 10 ng, about 20 ng, about 30 ng, about 40 ng, about 50 ng, about 100 ng, about 200 ng, about 300 ng, about 400 ng, about 500 ng, about 1 μg, about 5 μg, about 10 μg, about 20 μg, about 30 μg, about 40 μg, about 50 μg, about 100 μg, about 200 μg, about 300 μg, about 400 μg, about 500 μg, about 1 mg, about 5 mg, about 10 mg, about 20 mg, about 30 mg, about 40 mg, about 50 mg, about 100 mg, about 200 mg, about 300 mg, about 400 mg, about 500 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, or about 9,500 mg. Those of ordinary skill in the art of pharmaceutical formulation can readily deduce suitable unit doses for other compounds exhibiting TRPV1 receptor agonist activity, including rapid-onset compounds exhibiting TRPV1 receptor agonist activity and/or short acting compounds exhibiting TRPV1 receptor agonist activity. One of ordinary skill in the art of pharmaceutical formulation can also readily deduce suitable unit doses for other types of active agents that may be incorporated into a dosage form of the invention.

A therapeutically effective amount of a particular active agent administered to a given individual will, of course, be dependent on a number of factors, including the concentration of the specific active agent, composition or dosage form, the selected mode of administration, the age and general condition of the individual being treated, the severity of the individual's condition, and other factors known to the prescribing physician. However, one of skill in the art would readily recognize that the therapeutically effective amount of a particular active agent must be selected so as to allow for as-needed administration, as defined further herein.

With an immediate release dosage form, as-needed administration may involve drug administration immediately prior to when commencement of defecation would be desirable. The as-need administration can range from about 0 minutes to about 40 minutes prior to the desired emptying, from about 0 minutes to about 20 minutes prior, or about 0 minutes to about 5 minutes prior.

In another embodiment, a packaged kit is provided that contains the pharmaceutical formulation to be administered, i.e., a pharmaceutical formulation containing a therapeutically effective amount of a selected active agent for the treatment of defecation dysfunction, a container, preferably sealed, for housing the formulation during storage and prior to use, and instructions for carrying out drug administration in a manner effective to treat the defecation dysfunction. The instructions will typically be written instructions on a package insert and/or on a label. Depending on the type of formulation and the intended mode of administration, the kit may also include a device, for example, a syringe and plunger, with or without a rectal tip, for administering the formulation. The formulation may be any suitable formulation as described herein. The active agent can be a rapid onset and short acting TRPV1 receptor agonist, or a pharmaceutically acceptable salt thereof. The manner for treating the defecation dysfunction may be administration on an as-needed basis to treat the defecation dysfunction. The as-need basis can range from about 0 minutes to about 40 minutes prior to when the defecation is desired, from about 0 minutes to about 20 minutes prior to when the defecation is desired, or from about 0 minutes to about 5 minutes prior to when defecation is desired.

The kit may contain multiple formulations of different dosages of the same agent. The kit may also contain multiple formulations of different active agents. The kit may contain formulations suitable for sequential, separate and/or simultaneous use in the treatment of defecation dysfunction, and instructions for carrying out drug administration where the formulations are administered sequentially, separately and/or simultaneously in the treatment of defecation dysfunction. The parts of the kit may be independently held in one or more containers—such as bottles, syringes, plates, wells, blister packs, or any other type of pharmaceutical packaging—and may also include a device that can administer a specific dose, e.g., DIASTAT AcuDial, or calibrated syringe.

In another embodiment, a pharmaceutical formulation is provided for treating defecation dysfunction in a mammal in need of treatment on an as-needed basis, which includes a therapeutically effective amount of a rapid onset and short acting TRPV1 receptor agonist, or a pharmaceutically acceptable salt thereof, and a carrier for administration of the TRPV1 receptor agonist to the mammal on the as-needed basis. The pharmaceutical formulation can also be useful for inducing defecation in a mammal without an actual defecation dysfunction. The carrier for administration may be any suitable formulation as described herein. The as-need administration can range from about 0 minutes to about 40 minutes prior to when the defecation is desired, from about 0 minutes to about 20 minutes prior to when the defecation is desired, or from about 0 minutes to about 5 minutes prior to when the defecation is desired.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### General Methods: TRPV1 Receptor Agonists for On-Demand Defecation in Rat

The following methods were used to determine the efficacy, specificity, onset of action and the duration of action of various TRPV1 receptor agonists on gastrointestinal (GI) function in naïve rats. In addition, the reproducibility of multiple dosing and the pharmacodynamic (PD) responses of administering the TRPV1 receptor agonists via intrarectal administration were explored. In summary, TRPV1 receptor agonists produced dose-dependent increases in colorectal contractions consistent with use of TRPV1 receptor agonists for inducing defecation. The onset of action of the TRPV1 receptor agonist is rapid with a short duration. Repeated dosing of TRPV1 receptor agonists produced increases in colorectal pressure without significant reduction in response.

### Rat Methods:

General: In vivo studies were performed in anesthetized, spinally intact rats (N=10). Adult male Sprague Dawley rats (Charles River, 250-300 g) were housed at an AAALAC approved facility in cages (2-3/cage) with free access to water and food in a colony room that was maintained on a 12 hr light/12 hr dark cycle 1-2 weeks before the experiment. Rats were anesthetized with urethane (1.2-1.4 g/kg subcutaneous injection). Surgical procedures were then performed with the addition of isoflurane anesthesia (0.05-1.5% in O₂) as the full anesthetic effect of urethane takes about 1-2 hrs.

A catheter filled with heparinized saline (30 unit/ml) was inserted into the carotid artery and connected to a pressure transducer for measurement of blood pressure and heart rate. A catheter was inserted into the jugular vein for intravenous (i.v.) administration of drugs. Blood pressure and rectal pressure signals were amplified and displayed on a computer using LABCHART software (AD Instruments, Colorado Springs, CO).

Colorectal Contractility: Colorectal pressures were measured via a latex balloon catheter (length 2-3 cm) inserted (˜3-4 cm) into the distal colorectal region. The catheter was connected to a pressure monitoring system. The pressure in the balloon catheter was slowly increase to 10-15 mmHg by infusing saline (0.2-0.5 ml total volume) and this pressure was maintained throughout the study. This allowed drug induced changes in colorectal pressure to be monitored. Parameters measured include peak colorectal pressure response, duration of time above baseline activity (in the 1^{st} 5 min after drug administration), area under the curve (measured during the 1^{st} 5 min after drug administration) and the number of contractile events after vehicle and drug administration.

Defecation in awake animals was monitored in metabolism cages. Animals were habituated to handling and the metabolism cages on 2-4 occasions prior to dosing and were then randomized to a treatment arm. Rats were weighed and placed into metabolism cages for 10 min prior to intrarectal administration of vehicle or TRPV1 agonist. The number of fecal pellets and timing of events were recorded by trained personnel that were blinded to treatment. Fecal weight was weighed at 20 min post dosing.

Dosing: Intrarectal TRPV1 agonists were administered in a dose range of 0.01-2% wt/vol. Nonivamide, OEA, OLDA and capsaicin were dissolved in 95% ethanol at 20-100 mg/mL and then diluted to 0.01-2% with the vehicle (Tween-80:95% ethanol:saline in a ratio of 10:10:80) and administered intrarectally as a solution using a small gauge tube, or a solution soaked cotton wool plug, to mimic a suppository-like dosage form.

Data analysis: Data were examined qualitatively and quantitatively. The mean and standard error of the mean were calculated using MICROSOFT EXCEL. A Student's unpaired t-test was performed using PRISM 5 for WINDOWS (Graphpad Software, Inc., La Jolla, CA). A P value <0.05 was considered statistically significant.

### Example 2

### Intrarectal Nonivamide Solution (0.1%) Produced an Increase in Rectal Pressure in Anesthetized Naïve Rats

Methods: The TRPV1 receptor agonist nonivamide was administered to anesthetized spinal intact male rats (N=6) instrumented for rectal pressure recording via a balloon catheter inserted into the rectum 3-4 cm from the anus. A 0.1% solution (0.2 mL of a 1 mg/ml solution in Tween-80:ethanol:saline (ratio 10:10:80)) of nonivamide was administered via a 20 gauge feeding tube that was inserted ˜2 cm into the rectum. For comparison, vehicle (Tween-80:ethanol:saline in a ratio of 10:10:80) was administered using the same techniques.

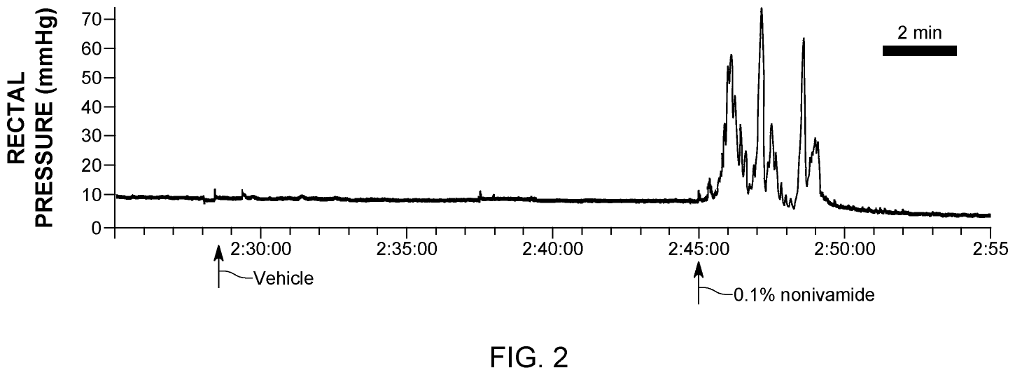

Results: Intrarectal administration of 0.1% nonivamide solution produced a transient rectal pressure increase in 6 of 6 rats with a rapid onset time of 1-2 min and short duration (5 min). Vehicle did not evoke any increase in rectal pressure. FIG. 2 shows a representative example of rectal pressure measured from a male rat. In FIG. 2, the black arrows indicate when vehicle or nonivamide solution (0.1%) was administered into the rectum.

Conclusions: Intrarectal nonivamide, but not vehicle, produced a transient increase in rectal pressure that demonstrated a rapid-onset and short duration.

### Example 3

### Intrarectal Nonivamide Solution (1%) Produced a Repeatable Increase in Rectal Pressure in Anesthetized Naïve Rats

Methods: The TRPV1 receptor agonist nonivamide was administered to anesthetized spinal intact male rats instrumented for rectal pressure recording via a balloon catheter inserted into the rectum 3-4 cm from the anus. A 1% solution (0.2 mL of a 10 mg/ml solution in Tween-80:ethanol:saline (ratio 10:10:80)) of nonivamide was administered via a 20 gauge feeding tube that was inserted 2-3 cm into the rectum.

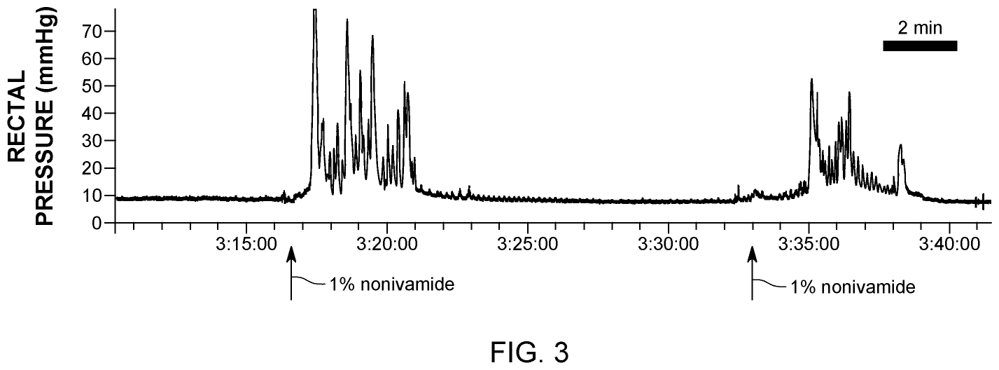

Results: Intrarectal administration of 1% nonivamide solution produced a transient rectal pressure increase with a rapid-onset time of 1-2 min and short duration (5-10 min). Repeat dosing of 1% nonivamide 17 min after the initial dose evoked a similar response. FIG. 3 shows a representative example of rectal pressure measured from a male rat. In FIG. 3, the black arrows indicate when nonivamide solution (1% in 0.2 mL of Tween-80:ethanol:saline in a ratio of 10:10:80) was administered into the rectum via a 20-gauge feeding tube catheter.

Conclusions: Intrarectal nonivamide produced a repeatable transient increase in rectal pressure that demonstrated a rapid-onset and short duration.

### Example 4

### Intrarectal Insertion of a Cotton Wool Plug, to Mimic a Suppository Dosage Form, Containing Nonivamide Produced an Increase in Rectal Pressure and Expulsion of Rectal Contents in Anesthetized Naïve Rats

Methods: Nonivamide was administered to a naive anesthetized male rat instrumented for rectal pressure recording via a balloon catheter inserted into the rectum 2-4 cm from the anus. To mimic a suppository dosage form, nonivamide (1%) was administered via a fluid-soaked small cotton wool plug. The cotton wool plug was placed into the anal canal at the distal rectum.

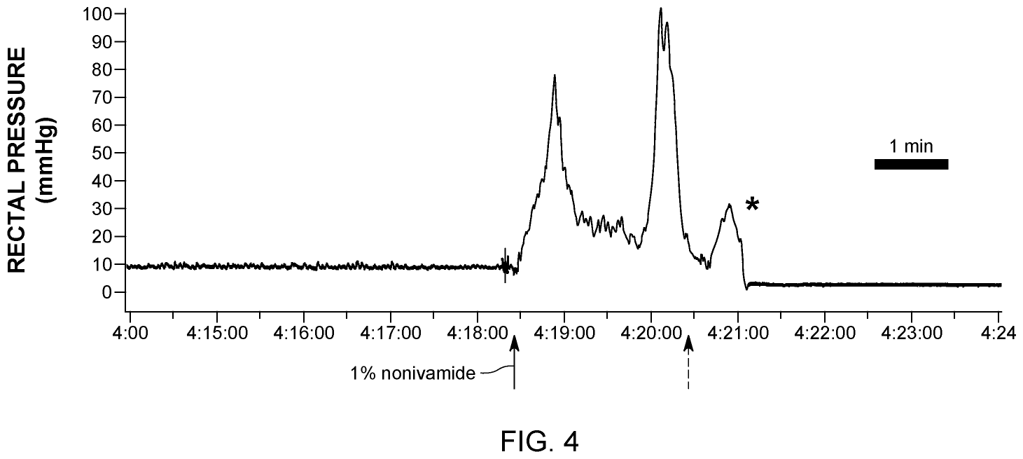

Results: Nonivamide produced a rapid-onset increase in rectal pressure within 1 minute of dosing. FIG. 4 shows a representative example of rectal pressure measured from 1 male rat. Nonivamide (small cotton wool plug soaked in 0.2 mL of 1% nonivamide in Tween-80:ethanol:saline in a ratio of 10:10:80) was administered into the rectum (black arrow in FIG. 4) and evoked a rectal response that was sufficient to expel the balloon catheter (* in FIG. 4). The open arrow in FIG. 4 indicates when the cotton wool plug was removed. The rectal balloon catheter was expelled from the rectum through the anus due to the nonivamide-induced increase in rectal pressure.

Conclusions: Intrarectal nonivamide produced a rapid-onset increase in rectal pressure of sufficient strength to expel rectal contents.

### Example 5

### Intrarectal OEA Produced a Rapid, Transient Increase in Rectal Pressure in Anesthetized Naïve Rats

The TRPV1 receptor agonist OEA produced an increase in colorectal pressure in experiments performed as described above.

Methods: OAE (N=4) was administered to anesthetized naïve male rats (N=5) instrumented for rectal pressure recording via a balloon catheter inserted into the rectum 3-4 cm from the anus. OEA was administered as 0.01-0.2% solution (0.2 mL of a 0.01-2 mg/ml solution in Tween-80:95% ethanol:saline (ratio 10:10:80)) via a small cotton wool plug soaked in 0.2 mL of solution, to mimic a suppository dosage form, that was inserted into the rectum. For comparison, vehicle was administered to all rats using the same techniques.

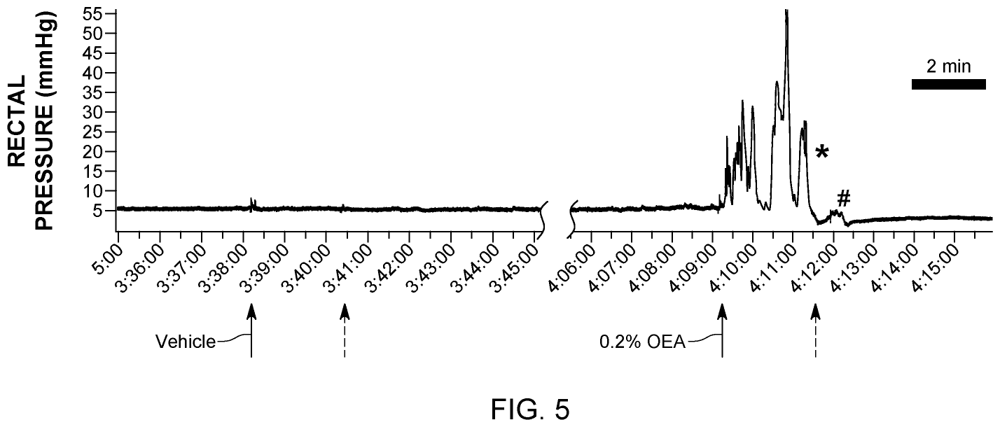

Results: Intrarectal administration of OEA produced a transient rectal pressure increase with a rapid-onset time of 0.5-2 min and short duration (5-10 min). Vehicle did not evoke any increase in rectal pressure. FIG. 5 shows a representative example of rectal pressure measured from a male rat that received intrarectal vehicle or 0.2% OEA. As shown in FIG. 5, vehicle (cotton wool plug soaked with 0.2 mL of Tween-80:ethanol:saline in a ratio of 10:10:80) administration (black arrow) did not change rectal pressure. OEA (cotton wool plug soaked in 0.2 mL of 0.2% OEA) was administered into the rectum (black arrow) and evoked a rectal response that was sufficient to expel the balloon catheter (*). In FIG. 5, the open arrows indicate when the cotton wool plug was removed, and the #symbol indicates defecation of fecal pellets.

Conclusions: Intrarectal OEA, but not vehicle, produced a transient increase in rectal pressure that demonstrated a rapid-onset and short duration. The increase in pressure was sufficient to expel the balloon catheter and induce defecation.

### Example 6

### Intrarectal OLDA Produced a Rapid, Transient Increase in Rectal Pressure in Anesthetized Naïve Rats

The TRPV1 receptor agonist OLDA produced an increase in colorectal pressure in experiments performed as described above.

Methods: OLDA (N=1) was administered to an anesthetized naïve male rat instrumented for rectal pressure recording via a balloon catheter inserted into the rectum 3-4 cm from the anus. OLDA was administered as 0.01-0.2% solution (0.2 mL of a 0.01-2 mg/ml solution in Tween-80:95% ethanol:saline (ratio 10:10:80)) via a small cotton wool plug soaked in 0.2 mL of solution, to mimic a suppository dosage form of OLDA, that was inserted into the rectum. For comparison, vehicle was administered using the same techniques.

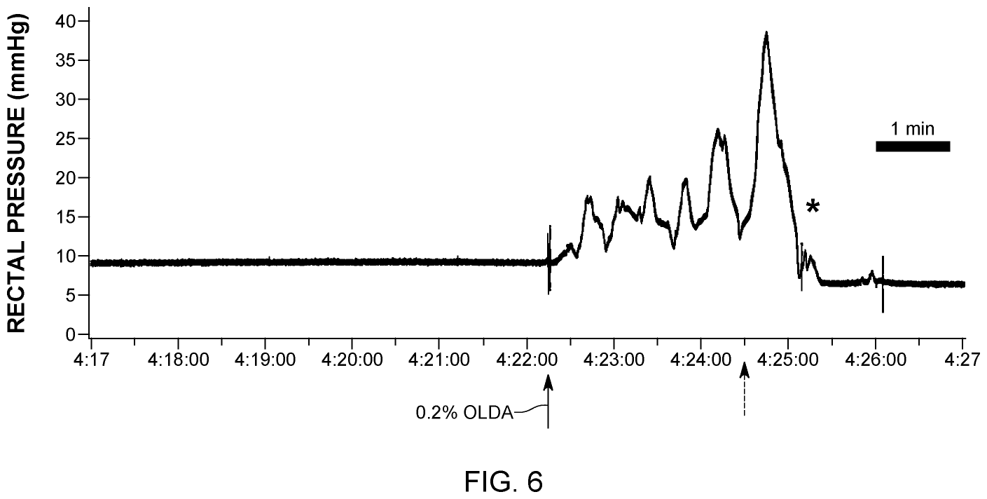

Results: Intrarectal administration of 0.2% OLDA produced a transient rectal pressure increase with a rapid-onset time of 0.5-2 min and short duration (5 min). Vehicle did not evoke any increase in rectal pressure. FIG. 6 shows an example of rectal pressure measured from a male rat that received intrarectal 0.2% OLDA. As shown in FIG. 6, the OLDA (cotton wool plug soaked in 0.2 mL of 0.2% OLDA in Tween-80:ethanol:saline in a ratio of 10:10:80) was administered into the rectum (black arrow) and evoked a rectal response that was sufficient to expel the balloon catheter (*). The open arrow indicates when the cotton wool plug was removed.

Conclusions: Intrarectal OLDA produced a transient increase in rectal pressure that demonstrated a rapid-onset and short duration and was sufficient in strength to expel the balloon catheter.

### Example 7

### Intrarectal Capsaicin Produced a Colorectal Pressure Increase of Sufficient Force to Induce Defecation in Conscious Spinal Intact Rats

Methods: Intrarectal capsaicin (N=3) was administered via a small cotton wool plug, to mimic a suppository dosage form of capsaicin, to anesthetized naïve male rats instrumented for rectal pressure recording via a balloon catheter inserted into the rectum 3-4 cm from the anus. Intrarectal capsaicin was administered to conscious rats (N=6) as 0.01-0.2% solution (0.2 mL of a 0.01-2 mg/ml solution in Tween-80:95% ethanol:saline (ratio 10:10:80)) using a small gauge tube that was inserted into the rectum. For comparison, vehicle was administered to all rats using the same techniques.

Defecation in conscious rats was monitored in metabolism cages. Rats were habituated to handling and the metabolism cages on 2-4 occasions before dosing, then randomized to the treatment arm. Rats were weighed and placed into metabolism cages for 10 min prior to receiving an intrarectal dose of vehicle or capsaicin. Since manual stimulation during intrarectal dosing in conscious rats can induce defecation, palpation of the distal rectum was performed to remove any fecal pellets located in the region of the anal sphincter. This manipulation resulted in removal of 1-3 fecal pellets that were near expulsion with concomitant contractions/relaxation of the anal sphincter. Once the anal sphincter had ceased contracting/relaxing (15-90 s) a fine catheter (22 G soft/flexible blunt tip catheter) was used to dose the vehicle or capsaicin, as this method produced little manual stimulation. Capsaicin was prepared as a 0.01, 0.1 or 1% solution (0.1, 1 or 10 mg/ml in tween-80/ethanol/saline (ratio of 10/10/80) or vehicle. Dose volumes were 0.1 mL and administered via the catheter inserted ˜1 inch through the anus into the rectum. The number of fecal pellets and timing of events were recorded by trained personnel blinded to the treatment. Fecal weight was recorded at 20 min post dosing.

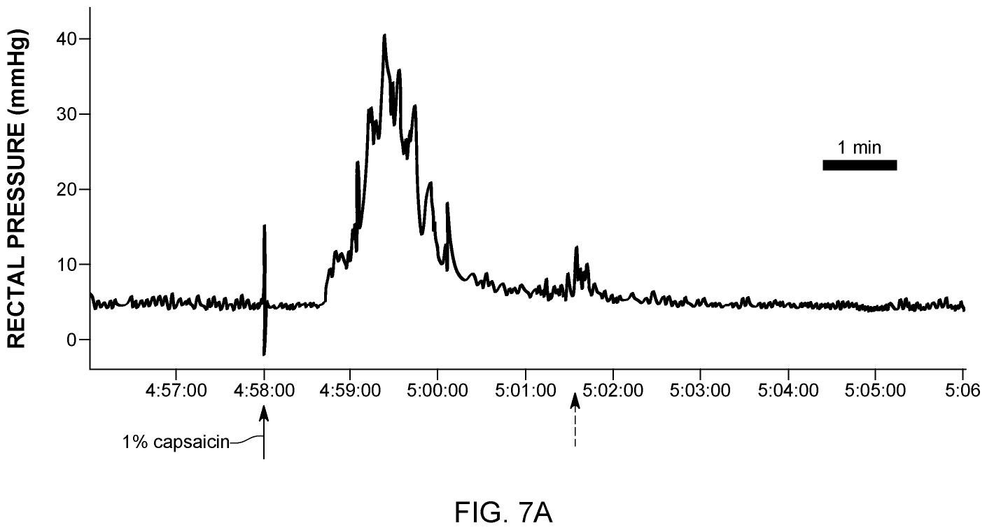

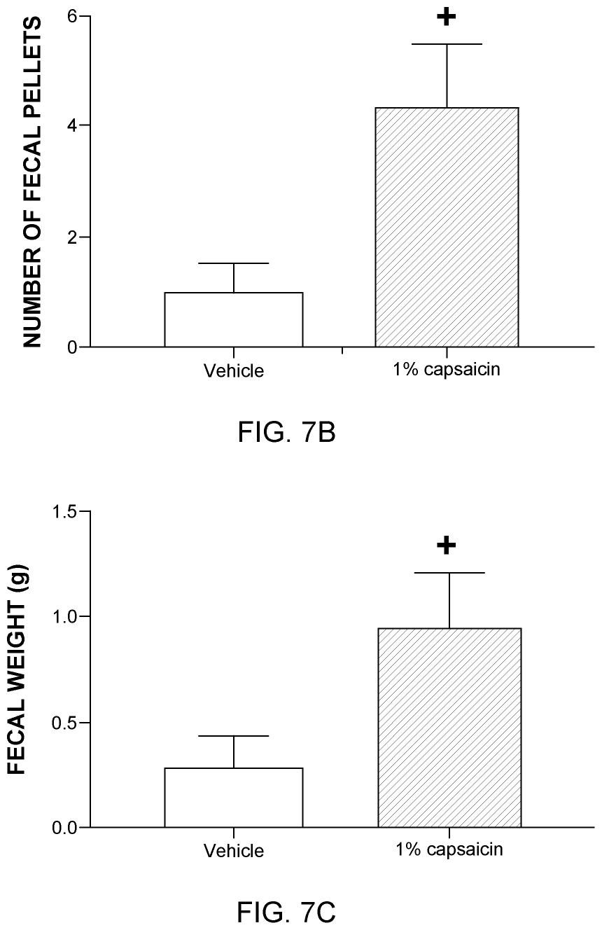

Results: Capsaicin induced an increase in colorectal pressure that was rapid in onset (<1 min) and short in duration (<5 min) in the anesthetized rat (FIG. 7A). In conscious awake freely moving rats, capsaicin significantly increased the number and total weight of fecal pellets expelled compared to vehicle (FIGS. 7B, 7C). Defecation occurred within 5 min and was complete within 10 min. FIG. 7A is an example of rectal pressure measured from a male rat that received intrarectal 1% capsaicin. As can be seen in FIG. 7A, capsaicin (1% soaked cotton wool plug) was administered into the rectum (black arrow) and a rapid, transient increase in colorectal pressure (peak pressure of ˜40 mmHg) was seen. The increase in pressure was sufficient to induce defecation in the awake rat. FIGS. 7B and 7C are charts illustrating induction of defecation. FIG. 7B shows the number of fecal pellets expelled in 10 min after vehicle (open bar) or 1% capsaicin (black bar). FIG. 7C shows the total expelled fecal weight 20 min after vehicle (open bar) or 1% capsaicin (black bar). The + symbol indicates a significant difference from the vehicle group (p<0.05, unpaired t-test). Additionally, FIGS. 7B and 7C show the mean and standard error of the mean, N=6 rats for vehicle and 6 rats for 1% capsaicin.

Conclusions: Capsaicin induced an increase in colorectal pressure in anesthetized rats of sufficient in strength to induce defecation in conscious naive rats.

### Example 8

### Additional Testing with Capsaicin was Performed. General Methods: Measurement of Capsaicin-Induced Rectal Responses

The following methods were used to determine the efficacy, specificity, onset of action and the duration of action of capsaicin to induce defecation in naïve rats and a rat model of chronic spinal cord injury. In addition, the reproducibility of multiple dosing and the pharmacodynamic (PD) responses of administering capsaicin via intrarectal administration were explored.

General: In vivo studies were performed in anesthetized, acute spinal cord injured (SCI) rats, anesthetized spinal intact rats, and awake chronic spinal cord injured rats. Adult female and male Sprague Dawley rats (Charles River, NC; 250-650 g) were housed at an AAALAC approved facility in cages (1-3/cage) with free access to water and food in a colony room that was maintained on a 12 hr/12 hr light/dark cycle.

For chronic SCI model preparation, animals were anesthetized with a mixture of ketamine/xylazine (50-100 mg/kg and 5-10 mg/kg intraperitoneal, respectively), the skin and muscle on the dorsal side at the level of the thoracic vertebrae were incised, and the spinal cord was carefully exposed by a laminectomy and transected at either T3-T4 or T8-T10 spinal level. Gelfoam^{R} was placed at the incision site and the muscle and skin overlying the vertebrae were closed with wound clips. The animal recovered from surgery for 7-10 weeks before being studied.

For the acute SCI model, animals were anesthetized with urethane (1.0-1.4 g/kg subcutaneous injection), the skin and muscle on the dorsal side at the level of the thoracic vertebrae were incised, and the spinal cord was carefully exposed by a laminectomy and transected at T8-T10 spinal level. Gelfoam^{R} was placed at the incision site and the muscle and skin overlying the vertebrae were closed with wound clips. The spinal cord was transected at least 60 min before starting the experimental protocol.

For all rectal contractility studies, animals were anesthetized with urethane (1.0-1.4 g/kg subcutaneous injection). Surgical procedures were then performed with the addition of isoflurane anesthesia (0.05-1.5% in O2) as needed. Rectal pressure signals were amplified and displayed on a computer using LABCHART (AD Instruments, Colorado Springs, CO).

Rectal Contractility: Rectal pressures were measured via a latex balloon catheter (length 1.5-4 cm) inserted (˜4 cm) into the distal rectal region. The catheter was connected to a pressure monitoring system. The pressure in the balloon catheter was slowly increased to 15-20 mmHg by infusing saline (0.2-0.5 ml total volume) and this pressure was maintained throughout the study, allowing drug-induced changes in rectal pressure to be monitored. Parameters measured included peak rectal pressure response, duration of time above baseline activity (in the 1^{st} 5 min after drug administration), area under the curve (measured during the 1^{st} 5 min after drug administration) and the number of contractile events after vehicle and drug administration.

Defecation in awake animals was monitored in metabolism cages. Animals were habituated to handling and the metabolism cages on 2-4 occasions prior to dosing and were then randomized to a treatment arm. Rats were weighed and placed into metabolism cages for 10 min prior to intrarectal administration of vehicle or capsaicin. The number of fecal pellets and timing of events were recorded by trained personnel that were blinded to treatment. Fecal weight is weighed at 10 and 30 min post dosing. Animals were re-tested 2-3 days later in a cross over design.

Dosing: Intrarectal capsaicin was administered in a dose range of 0.1-2% wt/vol. Capsaicin was dissolved in 95% ethanol at 50-100 mg/mL and then diluted to 2-20 mg/mL with either saline or water to form a suspension and administered intrarectally as a solution using a micropipette, blunt tip tube (22 G) or as a small solution-soaked cotton ball suppository (3-5 mm diameter). Vehicle doses were prepared using the same concentrations of ethanol in water or saline.

Data analysis: Data were examined qualitatively and quantitatively. The mean, standard deviation and standard error of the mean were calculated using MICROSOFT EXCEL. One way ANOVA tests were performed with PRISM 5 for WINDOWS (GraphPad Software, Inc., La Jolla, CA) and unpaired and paired t-tests were performed in EXCEL. P<0.05 is considered statistically significant.

### Example 9

### Intrarectal Capsaicin Solution Produced Transient, Repeatable Increased Rectal Pressure Responses in Anesthetized Naïve Rats

Methods: Capsaicin was administered to anesthetized spinal intact male and female rats (N=4 males and 1 female) instrumented for rectal pressure recording via a balloon catheter inserted into the rectum 2-4 cm from the anus. Capsaicin was administered as 0.2% solution (0.05-0.32 mL of a 2 mg/ml solution in 4.7 or 9.5% ethanol/saline) via a PE50 tube that was inserted into the rectum alongside the balloon catheter. For comparison, vehicle was administered to all rats using the same techniques.

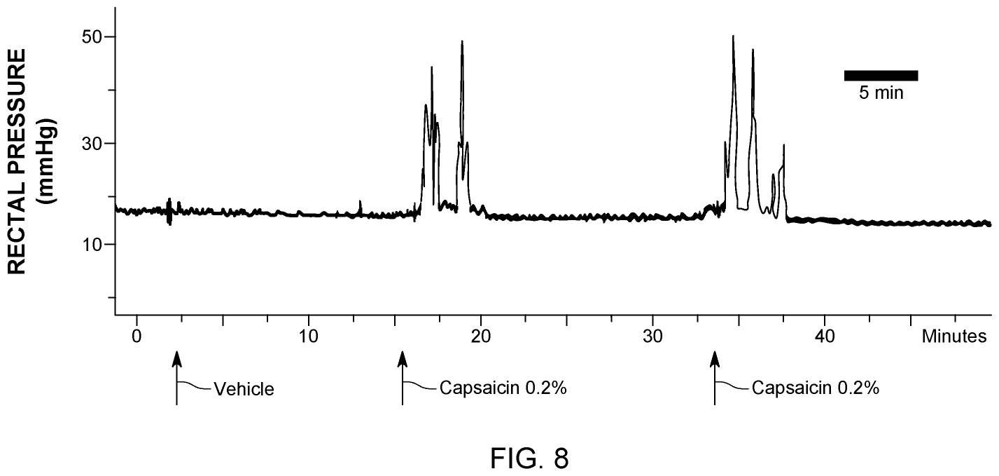

Results: Intrarectal administration of 0.2% capsaicin solution produced a transient rectal pressure increase in 5 of 5 rats with a rapid-onset time of 1-2 min and short duration (5 min). Vehicle did not evoke any rectal activity. FIG. 8 shows a representative example of rectal pressure measured from a male rat. Repeat dosing of 0.2% capsaicin 20 min after the initial dose evoked a similar response (FIG. 8). Administration of vehicle or capsaicin solution is indicated by arrows in FIG. 8. As illustrated by FIG. 8, intrarectal capsaicin produced a transient, repeatable increase in rectal pressure.

Conclusions: Intrarectal capsaicin, but not vehicle, produced a transient increase in rectal pressure that demonstrated a rapid-onset and short duration. The increase in rectal pressure was repeatable in the same animal.

### Example 10

### Intrarectal Insertion of a Cotton Ball Suppository Containing Capsaicin Produced Transient, Increased Rectal Pressure Responses in Anesthetized, Spinal Cord Injured (SCI) Rats

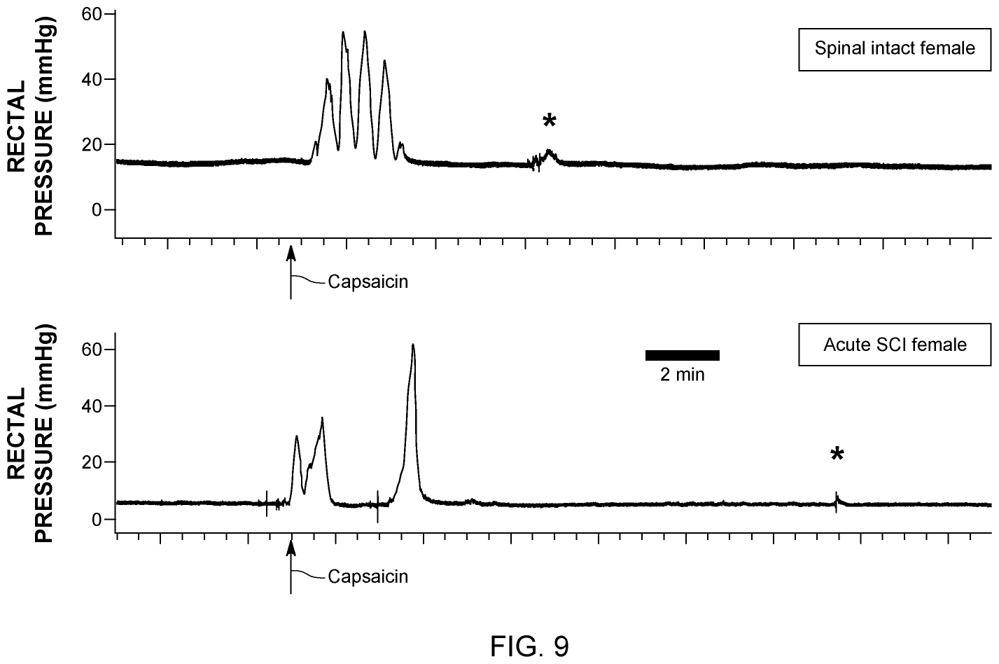

Methods: Capsaicin was administered to 2 anesthetized female rats instrumented for rectal pressure recording via a balloon catheter inserted into the rectum 2-4 cm from the anus. To increase the contact time of the capsaicin solution, capsaicin (0.2%) was administered via a fluid-soaked cotton ball suppository. A small cotton ball soaked in capsaicin (50 uL of a 0.2% solution) was administered into the rectum (filled arrows in FIG. 9). The cotton ball was placed into the anal canal at the distal rectum. The cotton ball was removed 5-12 min later (asterisks in FIG. 9). Top and bottom traces in FIG. 9 were recorded from individual female rats.

Results: A rapid-onset (<2 min) and short duration (<5 min) capsaicin-induced rectal pressure increase was observed in both rats. FIG. 9 shows representative examples of rectal pressure measured from 2 female rats. The top trace was recorded from an anesthetized spinal intact naïve female rat. The bottom trace was recorded from an anesthetized acute T9-transected SCI female rat. As shown, intrarectal administration of a capsaicin-soaked cotton ball produced rectal pressure responses in both rats similar to those produced by intrarectal administration of capsaicin solution. Placement of vehicle-soaked cotton ball produced either no response or a small increase in rectal activity, due to manual insertion of the cotton ball.

Conclusions: Capsaicin produced an increase in rectal pressure in spinal injured rats when administered using a locally restricted dosage form.

One skilled in the art will readily appreciate that the presently described subject matter is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference.
Callaghan, B., J. B. Furness and R. V. Pustovit (2018). “Neural pathways for colorectal control, relevance to spinal cord injury and treatment: a narrative review.” Spinal Cord 56(3): 199-205.
Fattori, V., M. S. Hohmann, A. C. Rossaneis, F. A. Pinho-Ribeiro and W. A. Verri (2016). “Capsaicin: Current Understanding of Its Mechanisms and Therapy of Pain and Other Pre-Clinical and Clinical Uses.” Molecules 21(7).
Hayashi, K., C. Shibata, M. Nagao, M. Sato, M. Kakyo, M. Kinouchi, F. Saijo, K. Miura, H. Ogawa and I. Sasaki (2010). “Intracolonic capsaicin stimulates colonic motility and defecation in conscious dogs.” Surgery 147(6): 789-797.
Jannin, V., G. Lemagnen, P. Gueroult, D. Larrouture and C. Tuleu (2014). “Rectal route in the 21st Century to treat children.” Adv Drug Deliv Rev 73: 34-49.
Kikuchi, D., C. Shibata, H. Imoto, S. Someya, T. Miyachi, K. Miura, T. Naitoh and M. Unno (2014). “Role of the intraluminal contents and the continuity of intrinsic neurons in intracolonic capsaicin-induced contraction and defecation in dogs.” Surg Today 44(1): 152-159.
MacDonagh, R., W. M. Sun, D. G. Thomas, R. Smallwood and N. W. Read (1992). “Anorectal function in patients with complete supraconal spinal cord lesions.” Gut 33(11): 1532-1538.
Matsumoto, K., E. Kurosawa, H. Terui, T. Hosoya, K. Tashima, T. Murayama, J. V. Priestley and S. Horie (2009). “Localization of TRPV1 and contractile effect of capsaicin in mouse large intestine: high abundance and sensitivity in rectum and distal colon.” Am J Physiol Gastrointest Liver Physiol 297(2): G348-360.
Nagano M, Ishimizu Y, Saitoh S, Okada H, Fukuda H. (2004). “The defecation reflex in rats: fundamental properties and the reflex center. Neurosci. 111(1):48-56.
Reyes-Escogido Mde, L., E. G. Gonzalez-Mondragon and E. Vazquez-Tzompantzi (2011). “Chemical and pharmacological aspects of capsaicin.” Molecules 16(2): 1253-1270.
Shafik, A. (1996). “Recto-colic reflex: role in the defecation mechanism.” Int Surg 81(3): 292-294.
Shafik, A. (1999). “A study of the effect of distension of the rectosigmoid junction on the rectum and anal canal with evidence of a rectosigmoid-rectal reflex.” J Surg Res 82(1): 73-77.
Shafik, A., O. El-Sibai and I. Ahmed (2001). “Role of the sigmoid colon in the defecation mechanism with evidence of sigmoido-anal inhibitory and ano-sigmoid excitatory reflex.” Front Biosci 6: B25-29.
van Wanrooij, S. J., M. M. Wouters, L. Van Oudenhove, W. Vanbrabant, S. Mondelaers, P. Kollmann, F. Kreutz, M. Schemann and G. E. Boeckxstaens (2014). “Sensitivity testing in irritable bowel syndrome with rectal capsaicin stimulations: role of TRPV1 upregulation and sensitization in visceral hypersensitivity?” Am J Gastroenterol 109(1): 99-109.
Ansel's Pharmaceutical Dosage Forms and Drug Delivery Systems 9th Edition by Lloyd V. Allen, Nicholas G. Popovich, Howard C. Ansel 2011

1. A method for inducing defecation in a mammal, which comprises administering to the mammal a therapeutically effective amount of transient receptor potential vanilloid 1 (TRPV1) receptor agonist or a pharmaceutically acceptable salt thereof, wherein the administering is by an intrarectal mode, wherein the TRPV1 receptor agonist or the pharmaceutically acceptable salt thereof, to induce the defecation.

2. A method for inducing defecation in a mammal, which comprises administering on an as-needed basis to the mammal a therapeutically effective amount of a TRPV1 receptor agonist, or a pharmaceutically acceptable salt thereof, formulated as an immediate release dosage form, wherein the administering is by an intrarectal mode, to induce the as-needed defecation.

3. The method of claim 1, wherein the intrarectal administering comprises a rectal suppository, ointment, cream or solution.

4. The method of claim 1, wherein the mammal is a human, a cat, or a dog.

5. The method of claim 1, wherein the rapid-onset is characterized by an onset time ranging from about 0 sec to about 10 minutes after TRPV1 receptor agonist administration.

6. The method of claim 1, wherein the rapid-onset is characterized by an onset time ranging from about 0 sec to about 5 min after TRPV1 receptor agonist administration.

7. The method of claim 1, wherein the short duration of action is characterized by a duration of action ranging from about 1 hour to about 10 minutes after TRPV1 receptor agonist administration.

8. The method of claim 1, wherein the short duration of action is characterized by a duration of action ranging from about 30 minutes to about 10 minutes after TRPV1 receptor agonist administration.

9. The method of claim 1, wherein the short duration of action is characterized by a duration of action ranging from about 15 minutes to about 1 minute after TRPV1 receptor agonist administration.

10. The method of claim 1, wherein the TRPV1 receptor agonist, or the pharmaceutically acceptable salt thereof, comprises nonivamide, OEA, or OLDA.

11. (canceled)

12. (canceled)

13. (canceled)

14. The method of claim 1, wherein the as-needed administering ranges from about 0 minutes to about 10 minutes prior to when the defecation is desired.

15. The method of claim 1, wherein the mammal is a female, with or without a neurological condition, such as spinal cord injury.

16. A method for treating defecation dysfunction in a mammal in need of treatment, which comprises administering on an as-needed basis to the mammal a therapeutically effective amount of a transient receptor potential vanilloid 1 (TRPV1) receptor agonist or a pharmaceutically acceptable salt thereof formulated as an immediate release dosage form, wherein the administering is by an intrarectal mode, wherein the TRPV1 receptor agonist or the pharmaceutically acceptable salt thereof, has a rapid onset and a short duration of action, to induce the as-needed defecation.

17. The method of claim 16, wherein the defecation dysfunction is idiopathic and/or a result of one of spinal cord injury, traumatic brain injury, multiple sclerosis, spina bifida, degenerative brain disease, Alzheimer's, Parkinson's, dementia, diabetes, advanced age, or postoperative status, chronic idiopathic constipation, and irritable bowel syndrome with constipation, drug-induced constipation (e.g. opioids, antimuscarinics), or other forms of constipation, or combinations thereof.

18. The method of claim 16, wherein the TRPV1 receptor agonist, or the pharmaceutically acceptable salt thereof, comprises nonivamide, OEA, or OLDA.

19. (canceled)

20. (canceled)

21. The method of claim 16, wherein the intrarectal administering comprises a rectal suppository.

22. The method of claim 16, wherein the mammal is a human, a cat, or a dog.

23. The method of claim 16, wherein the rapid-onset is characterized by an onset time ranging from about 0 sec to about 10 minutes after TRPV1 receptor agonist administration.

24. The method of claim 16, wherein the short duration of action is characterized by a duration of action ranging from about 1 hour to about 10 minutes after TRPV1 receptor agonist administration.

25. The method of claim 16, wherein the as-needed administering ranges from about 0 minutes to about 10 minutes prior to when the defecation is desired.
